(19) United States
(12) Patent Application Publication
(10) Pub. No.: US 2012/0021373 A1
(43) Pub. Date: Jan. 26, 2012

(54) Multi-Purpose Dental Instrument

(76) Inventor: Jackelyne Moreno, Euless, TX (US)

(21) Appl. No.: 13/186,622

(22) Filed: Jul. 20, 2011

Related U.S. Application Data
(60) Provisional application No. 61/366,433, filed on Jul. 21, 2010.

(51) Int. Cl. A61B 1/247

(52) U.S. Cl. 433/31

(57) ABSTRACT

A dental mirror tool having a body comprises a manifold disposed on the tool body proximate the mirror, a plurality of output ports disposed in an outlet side of the manifold and disposed to emit streams of light and fluid and air or suction toward a predetermined target, and a selection system for selecting the light, fluid, or air or suction emitted toward the predetermined target. In another aspect, a method for directing streams of light, fluid, air, or suction toward a target during a dental procedure, comprising the steps of attaching a manifold having output orifices for light, fluids, air, or suction to a body of the tool; connecting the orifices to sources of light, fluid, air, or suction; and controlling a substance emitted from the orifices to be directed toward the target.

### 1. CROSS-REFERENCE TO RELATED APPLICATION

The present application claims priority from Provisional Application Ser. No. 61/366,433, filed Jul. 21, 2010, entitled “Multipurpose Dental Instrument” by inventor, Jackelyne Rodriquez (Moreno).

### BACKGROUND OF THE INVENTION

2. Field of the Invention

The present invention generally relates to dental equipment and more specifically to handheld dental instruments for illumination, viewing, and cleaning a patient's teeth.

3. Background of the Invention and Description of the Prior Art

Conventional instruments or implements for use in dental care include mirrors, lamps, compressed air jets, and water jets or hydrants. Dental mirrors are typically small, round mirrors attached at an angle to the end of an elongated rod-like handle. The angle of the mirror with the handle may typically be in the range of 20 to 60 degrees relative to the longitudinal axis of the elongated handle. Such placement facilitates the dentist or assistant's observation of a patient's upper teeth while standing or seated in an erect position, thus minimizing possible discomfort and awkward viewing angles.

Compressed air and water jets are each typically provided as separate implements because they are supplied from different reservoirs, the compressed air from an air compressor and storage tank for example, and the water from a water pump and storage tank. Both air and water supplies are fed via a hose connected to the respective storage tank, and a hand held control handle or foot-operated control. Illumination of a patient's mouth is provided by a specialized overhead lamp fixture attached to a gimble assembly supported by a counterbalanced and articulated arm that may be positioned at will by the dentist or dental assistant. The lamp may be equipped with a lens to minimize scattered light and to focus the light on the work area of interest.

There are a number of disadvantages to this configuration of the dental instruments, both to the dentist and the patient. In many cases an assistant is required to hold and manipulate one or more of the dental implements while the dentist is at work performing dental treatment of or repair to a patient's teeth. In civilian or military field treatment, outside a dentist's office, handling the necessary implements can be even more awkward and cumbersome because they cannot easily supported or held except by the dental personnel themselves. For example, the dentist may need to manipulate simultaneously a drilling apparatus and a mirror, while an assistant manipulates a stream of water or air at the dentist's instruction. Illumination from a suspended lamp may provide the necessary light; however, to ensure maximum light upon the work area often requires that the hands and implements in use be positioned so as not to create shadows in the area of interest. Such positioning may require awkward, uncomfortable, or less than optimum orientation of the other implements to avoid blocking the light. Further, up to four hands may be in close, possibly interfering positions in order to properly manipulate the implements for a particular operation, resulting in compromised positioning, making the performance of the operation less efficient and more cumbersome, thus prolonging the time to perform the operation and increased discomfort to the patient.

Other disadvantages include, obviously, the discomfort to the patient due to the plurality of hardware implements positioned in and moving around in the patient's mouth. Further, the more such implements, the more widely opened the lower jaw of the patient must be, which often tends to increase the discomfort experienced by the patient. Moreover, the procedural steps performed by the dental personnel must be designed or modified to overcome the inefficiencies and inconveniences associated with the use of the plurality of conventional dental implements or instruments as described.

What is needed is a solution to the problems presented by the use of a plurality of dental instruments in treating dental patients. Making the implements lighter in weight, or even smaller than current practice, while offering mitigation of some of the difficulties associated with conventional dental tools, presents only a small step forward in solving these problems.

### SUMMARY OF THE INVENTION

Accordingly, there is provided by the present invention a dental instrument or tool that solves the aforementioned problems. The result is increased efficiencies and comfort, for both dental personnel and patients. These benefits, which provide an advancement in the state of the art, arise from the basic concept: to reduce the plurality of separate dental implements or tools that must be present in the patient's mouth at one time by combining the functions formerly provided by a plurality of single-function tools into one tool that is designed to provide all of the necessary functions. The result is a multi-function dental tool or instrument that provides a mirror, a beam of light, streams of water (or other fluid) and air, or a suction device, and that is also light in weight and easily maneuverable. The multi-function dental tool described herein reduces the number of different, separate tools that must be present in the patient's mouth at one time and frees up the number of tools that must be held and manipulated by the dental personnel. Another way of stating the latter advantage is that fewer hands are required to manipulate the dental tools necessary for performing many routine dental treatment operations.

In one embodiment, for use with a dental mirror tool having a body and a mirror attached to an end thereof and disposed at an angle to a longitudinal axis of the tool body, an apparatus, comprises a manifold disposed on the tool body proximate the mirror; a plurality of output ports disposed in an outlet side of the manifold, the ports positioned to emit streams of light and fluid and air toward a predetermined target; and a selection system disposed in the tool for selecting the light, fluid, or air emitted toward the predetermined target.

In another embodiment, a method for use with a single dental tool provides streams of light, air, and fluid directed toward a target during a dental procedure, comprising the steps of: attaching a manifold having output orifices for air, fluids, and light to a body of the tool; connecting the orifices to sources of said air, fluid, and light; and controlling a substance emitted from the orifices to be directed toward the target.

In yet another embodiment, a manifold for attaching to a handle of a dental tool having an angled mirror disposed on a first end thereof is provided, the manifold comprising a plurality of air, fluid, and light emission ports aimed at predetermined regions relative to the dental tool.

### BRIEF DESCRIPTION OF THE DRAWINGS

FIG. 1 illustrates a first perspective view of one embodiment of a multi-function dental tool according to the present invention;

FIG. 2 illustrates a second, partially disassembled perspective view of the embodiment of FIG. 1;

FIG. 3 illustrates an exploded view of the removable head piece portion of the embodiment of FIG. 2;

FIG. 4 illustrates an exploded view of the main body portion of the embodiment shown in FIG. 2;

FIG. 5 illustrates an internal cross section view from a first side of the embodiment of FIG. 1;

FIG. 6A illustrates a first side cross section view of the removable head piece portion of the embodiment of FIG. 2 showing an air and water tube directed toward the mirror;

FIG. 6B illustrates a second side cross section view of the removable head piece portion of the embodiment of FIG. 2 showing an air and water tube directed toward a patient's teeth;

FIG. 6C illustrates a third side cross section view of the removable head piece portion of the embodiment of FIG. 2 showing a light path directed toward a patient's teeth;

FIG. 7A illustrates a side cross section view of the control body sub-assembly portion of the main body of FIG. 5;

FIG. 7B illustrates a bottom view of the control body shown in FIG. 7A;

FIG. 7C illustrates an underside view of the control body chassis assembled with a retaining collar and wiring for the light assembly; and

FIG. 8 illustrates an electrical circuit diagram of the light source portion of the embodiment of FIGS. 4 and 5.

### DETAILED DESCRIPTION OF THE INVENTION

In the following description, reference numbers identifying the same structures are repeated in the successive drawings. The drawings illustrate one complete, preferred embodiment that utilizes the principles and concepts of the present invention set forth in the appended claims.

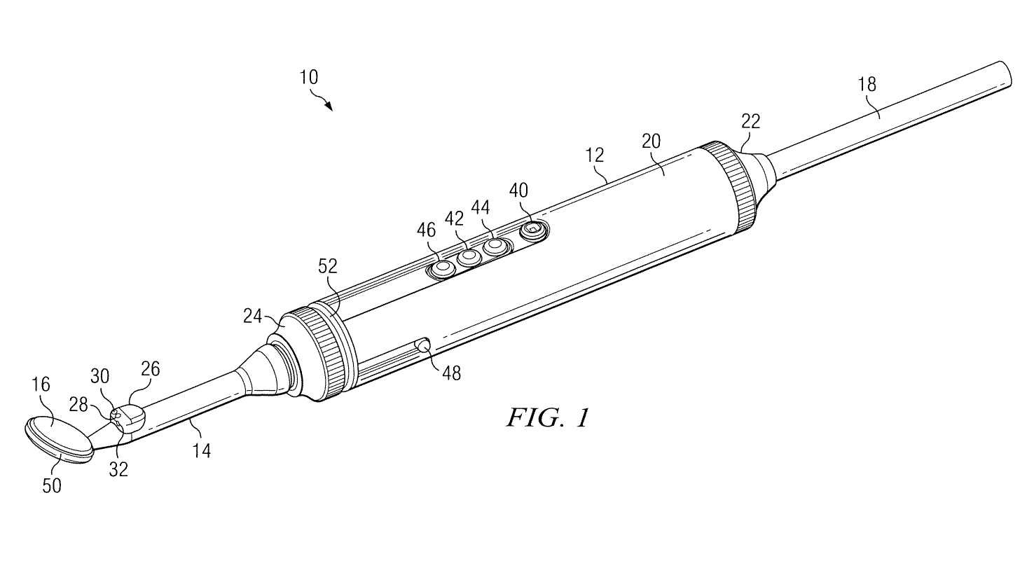

FIG. 1 illustrates a first perspective or isometric view of one embodiment of a multi-function dental tool 10 according to the present invention. The dental tool 10 includes a main body 12, a removable head piece 14, a mirror 16, and a supply line 18 having internal tubes connected to sources of fluid such as water, and air or vacuum. An air supply line may be connected to an air compressor (not shown) or a vacuum pump (not shown). The fluid supply line may typically be connected to a water supply such as a storage tank or mains system (not shown). The main body 12 of the dental tool 10 preferably includes an outer sleeve 20. A main body cap 22 is disposed at a rearward end of the main body 12, which encloses a connection interface and may also function as a strain relief for the supply line 18. A retaining collar 24 is disposed at a forward end of the main body 12, which secures the removable head piece 14 to the main body 12 as will be described.

The removable head piece 14 includes a fluid/light manifold 26 attached to and integrated with the head piece 14. The fluid/light manifold 26 provides a termination structure for the passages conducting fluid (e.g., water), air (either under pressure or a vacuum), and light to the outlet end of the dental tool 10 that provides the primary functionality of the tool. This manifold 26 includes the necessary orifices to provide outlets for fluids such as water and air, and a light beam output. For example, a first fluid/air orifice 28 may provide an outlet for a stream of air or water aimed toward a tooth of interest. A second fluid orifice 30 may provide an outlet for another stream of air or water aimed at the surface of the mirror for cleaning the mirror surface. It should be appreciated that, while water is contemplated for use in the dental tool 10 as described herein, fluids other than water may be used in certain treatment operations. A third light output orifice 32 may provide an outlet for a beam of light aimed at the surface of the mirror 16 to provide for illuminating a tooth or other structure of interest within a patient's mouth. The angles of the fluid, air and light outputs may be configured to suit particular applications for directing these materials or beams toward targets of interest.

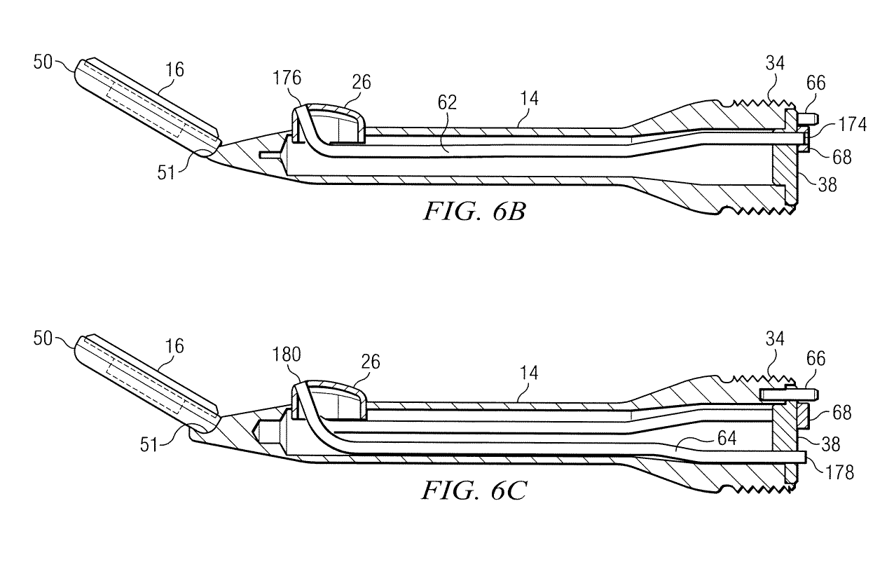

A mirror 16 may be supported by an angled mirror holder 50 of the removable head piece 14. The mirror, as depicted in FIG. 6A is preferably supported at an angle Θ of approximately 30 degrees with the longitudinal axis C_{L} of the main body 12 of the dental tool 10. This angle permits a dentist, hygienist, or assistant to enable viewing a patient's upper teeth without undue discomfort. However, other angles may be used, for example within a range of 10 to 60 degrees. In some embodiments of the dental tool 10 this angle may be adjustable. The angle O is shown in FIG. 6A.

A principle concept of the present invention—to provide multiple functions useful to a dentist or other dental care worker in a single implement—is made possible by the fluid/light manifold 26 component that is attached to or made integral with the body of a dental mirror tool proximate or nearby the mirror. This configuration enables embodiments that can direct streams of fluid (e.g., water) or air (either an air jet or an air suction or vacuum) or a light beam either toward the mirror or toward a target region in a patient's mouth in the vicinity of the mirror. Supplies of the fluid, air, and light may be provided for in the body of the multi-function dental tool and easily controlled using buttons accessible on the external surface of the tool that are placed to be readily operated with one hand. Fluids and air may be supplied to the tool from an external or portable source through a supply line or hose connected to the tool. It should be appreciated that the external source may be compact and portable to facilitate use of the dental tool in both civilian and military field situations because of the high utility of the multiple functions provided in the tool. In a preferred embodiment the tool requires no external connection to electricity because of its battery operation that energizes the light source within the tool. However, in alternate embodiments, a connection to an external source of electricity may be advantageously provided.

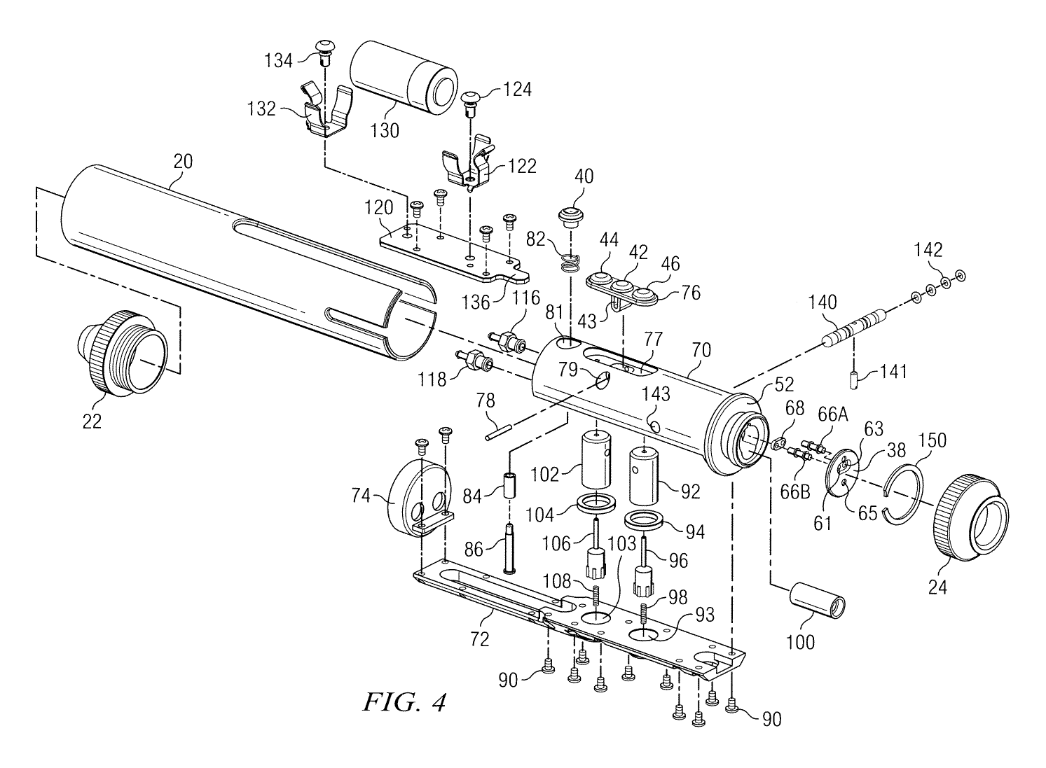

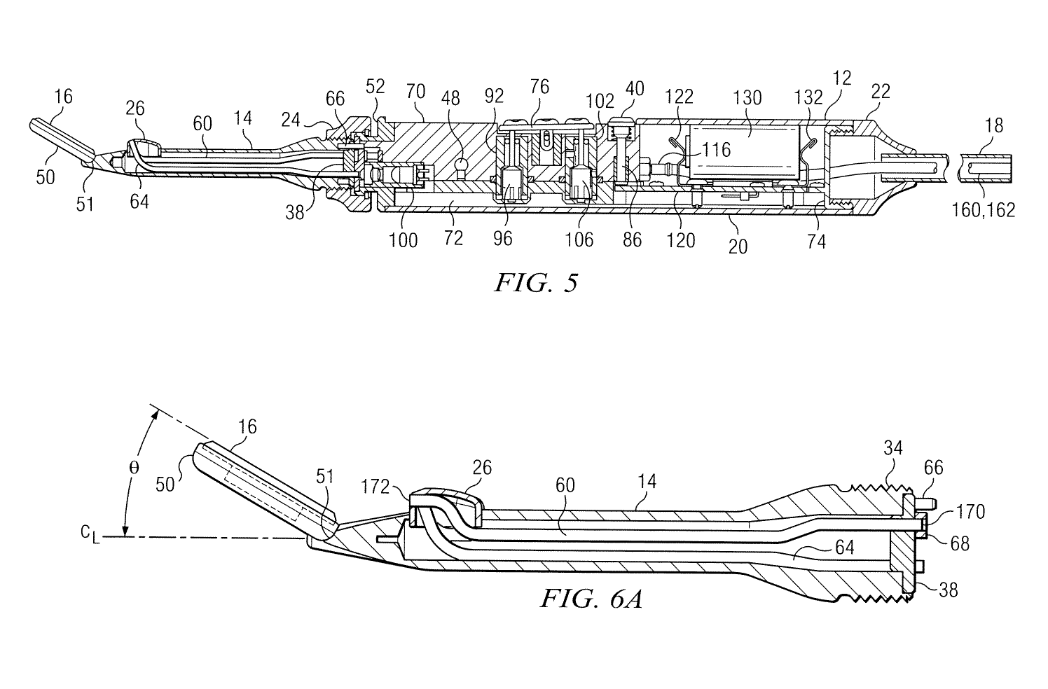

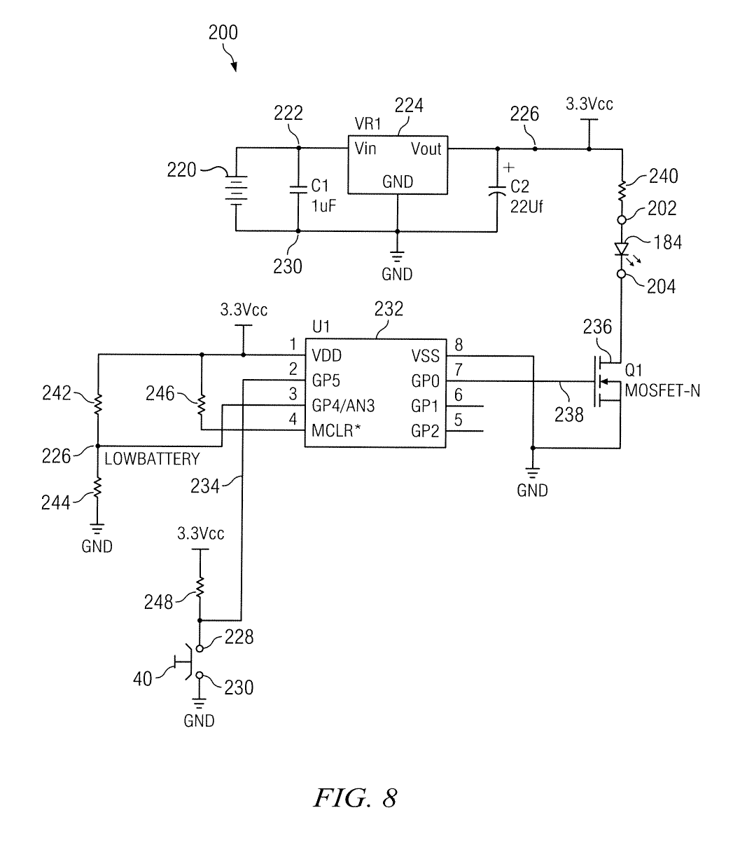

Continuing with FIG. 1, a row of control buttons is disposed along an upper side of the main body 12. Button 40 controls a light supply circuit (See FIG. 8) to provide the light output from the light output orifice 32. Buttons 42, 44, and 46 are configured as a bar that tilts about a pivot axis directly below the center button 42, to provide control of the flow of air and/or fluid, either individually or combined as will be described in FIGS. 4 and 5. A selector bar 48, for selecting the direction of fluid or air to be emitted from the fluid/light manifold 26 toward the mirror 16 or toward the patient's teeth, is seen protruding from the side of the main body 12. In operation, the selector bar 48 is pressed from one side of the main body 12 to the opposite side to direct the flow via one passage or the other, that is, toward the mirror or toward a tooth. An opening 140 in a center portion of the selector bar 48 acts as a valve for directing the flow of fluid—air and/or water, for example. See FIGS. 4 and 5 for internal details of this structural feature.

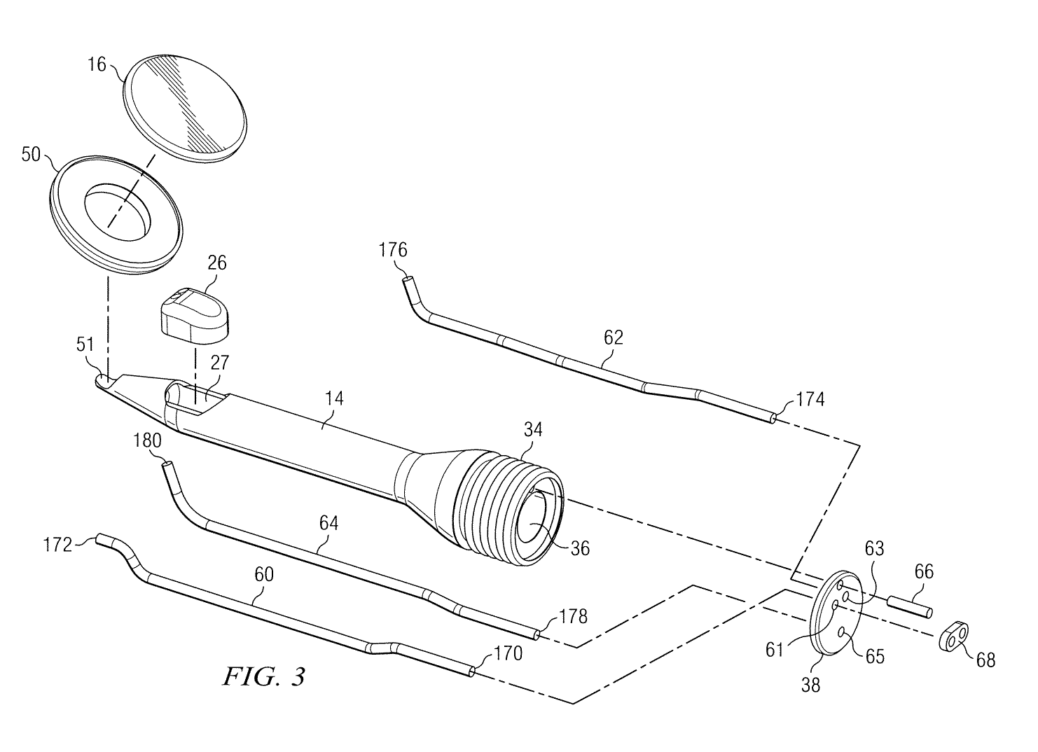

Materials preferred for use in fabricating most of the parts used in the dental tool 10 are generally either stainless steel, such as “alloy 304,” a readily available alloy of steel, chromium and nickel, or an FDA (U.S. Food and Drug Administration)-compliant plastic material, such as nylon. Generally, an engineering plastic suitable for high quality, precision manufactured small parts that can withstand repeated exposure to high temperatures (above 100 degrees Centigrade), which is FDA-compliant and meets the requirements for use in dentistry may be considered. In particular, the removable headpiece 14 must be able to withstand the heating of an autoclave used for sterilizing the dental tool 10 before each use, and be manufacturable with threads (See FIG. 3) that are sufficiently durable and accurate to enable many cycles of removal from and secure re-attachment of the head piece 14 to the main body 12. The material(s) selected may be fabricated by conventional methods for manufacturing precision parts, alone or used in combination, such as casting, machining or injection molding. These same materials are suitable for the rest of the solid or rigid parts. Materials referred to herein as “stainless steel” or “plastic” will be understood to mean the materials described in this paragraph unless otherwise noted. Further, as will be described, the dental tool 10 also contains several metal springs and a number of resilient sealing components fabricated from an FDA-compliant elastomer. The mirror 16 may be fabricated of glass with mirror finish or Rhodium-plated stainless steel. Persons skilled in the art will be familiar with suitable manufacturing methods for the embodiment described herein.

The versatile multi-function dental tool illustrated in FIG. 1 provides, in a single instrument a dental mirror, a light beam, an oral irrigation unit, an oral suction unit and may include, in an alternate embodiment through the use of an interchangeable head piece, an oral air jet unit for drying moist surfaces or clearing fluid or debris. The interchangeable head piece may also be used to enable the fluid or air to be directed alternately toward the mirror or toward a patient's teeth. Other combinations of the functional dental tools, simply by re-arranging the tubes in the head piece with ones of the appropriate configuration may be realized by persons skilled in the art. The remaining figures will illustrate and describe the various features and construction of the invention.

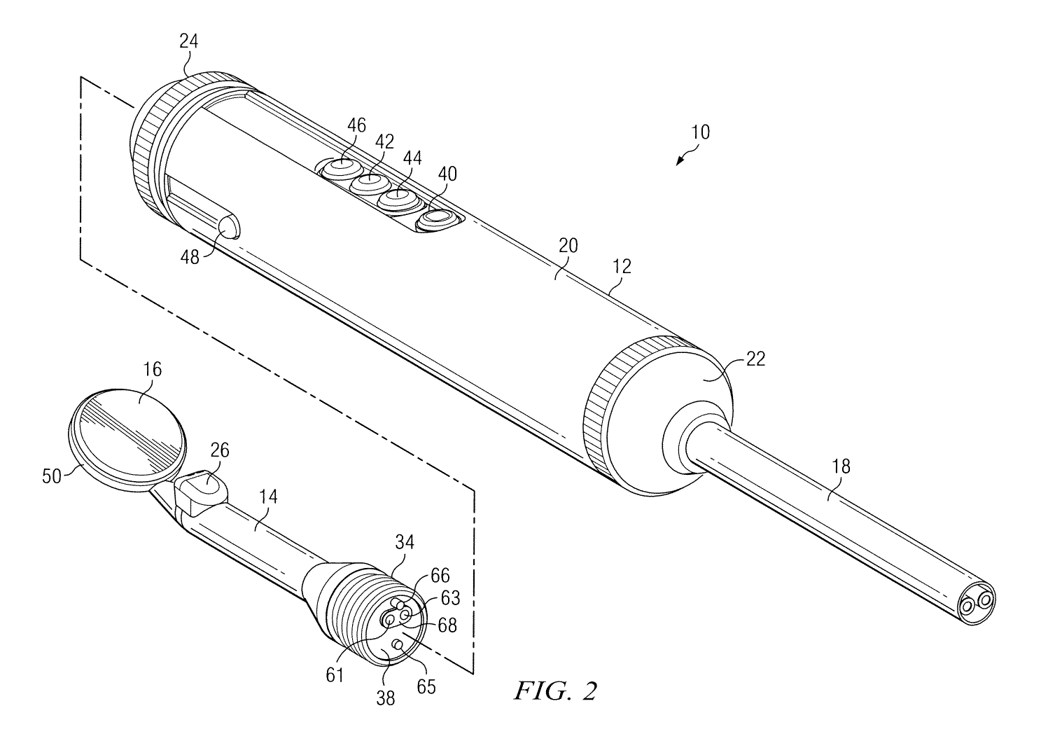

FIG. 2 illustrates a second, partially disassembled perspective view of the embodiment of FIG. 1 with the removable headpiece 14 removed from the main body 12 and exposing the bulkhead 38 within the proximate end of the headpiece 14. The bulkhead 38, to be described further in FIGS. 3 and 4, includes supporting passages 61, 63 for the air/fluid tubes 60, 62 (See FIG. 3) and 65 for the light tube 64 to pass through from the control body 72 within the main body 12 to the head piece 14. The passages 61 and 63 are supported and sealed in the bulkhead 38 by an elastomer seal 68. An alignment pin 66 to be described is also shown in FIG. 2. The remaining structures shown in FIG. 2 are as described in FIG. 1. The removable headpiece 14 is secured in the present example by the retaining collar 24, which is itself retained by a lip formed into the end of the control body 70, to be described as shown in FIG. 5. The head piece 14 is secured to the control body 70 by rotating the retaining collar 24 around the external threads 34 formed in the aft end of the removable headpiece 14. Alignment of the head piece 14 and the control body 70 is keyed by an alignment pin 66 shown in FIG. 3, further described in FIGS. 5 and 6A. As the headpiece 14 is drawn into contact with the control body 70, the passages 61, 63, and 65 will align with corresponding passages in the control body 70.

FIG. 3 illustrates an exploded view of the removable head piece 14 of the embodiment of FIG. 2. In addition to the structures previously described, which bear the same reference numbers, the drawing depicts several internal parts of the illustrative embodiment, including the tubes for conducting air or fluid and light between the bulkhead 38 and the fluid/light manifold 26. A first fluid tube 60 includes an inlet end 170 that connects to the bulkhead 38 at an opening 61 and an outlet end 172 that connects to a first fluid (or air) outlet orifice 28 in the fluid/air/light manifold 26. A second fluid tube 62 includes an inlet end 174 that connects to the bulkhead 38 at an opening 63 and an outlet end 176 that connects to a second fluid (or air) outlet orifice 30 in the fluid/light manifold 26. A third light tube 64 includes an inlet end 178 that connects to the bulkhead 38 at an opening 65 and an outlet end 180 that connects to a third light outlet orifice 32 in the fluid/light manifold 26.

The bulkhead 38 and the first 60 and second 62 fluid tubes may be fabricated of stainless steel or FDA-compliant plastic as mentioned previously. The light tube assembly 64 may include a stainless steel sheath that contains a fiber optic strand or bundle of strands for conducting light emitted by a light assembly 100 to be described in FIG. 4. An alignment pin 66 may be used to facilitate alignment of each of the fluid and light tubes through the bulkhead 38 structure as the head piece 14 is connected to the control body 70. The alignment pin 66 may be disposed in an upper portion of the bulkhead 38 in the illustrated example.

The tubes 60, 62 and 64 includes output ends that are directed respectively to the mirror 16, and to a region in a patient's mouth, i.e., away from the mirror. It should be understood that the output ends of these tubes may be oriented differently than illustrated in FIG. 3. It is possible that the removable head piece 14 may be configured with several different combinations of the output orientations of the tubes 60, 62, and 64 to provide a set of head pieces to suit particular applications. The removable head piece 14 thus may function as an interchangeable part of the multi-function dental tool of the present invention.

Continuing with the exploded view in FIG. 3, the mirror 16 is shown detached from the mirror holder 50 and the mirror holder 50 is shown as a separate structure that may be attached to a seat 51 formed in the nose end of the removable head piece 14. The mirror 16 may be provided as, for example, a Rhodium-plated stainless steel component, or a more conventional glass with silvered backing. However, a disadvantage of the latter is the parallax error introduced by a mirror of clear material backed by a reflective surface. Other equivalent reflective surfaces that can withstand the stresses placed upon the materials and structure thereof may be used. The mirror 16 may be secured in the mirror holder by welding or bonding or press fit according to the particular materials used, as will be understood by persons skilled in the art. The mirror holder 50 may be assembled to the seat 51 by welding or using an epoxy adhesive or equivalent cement capable of withstanding the temperatures of an autoclave and the nominal mechanical stress of handling and manipulating the dental tool. In some applications, particularly those where interchangeable mirrors might be included, the mirror holder may be supported by a threaded engagement with the nose portion of the head piece 14. The seat 51 may be configured to support the mirror holder 50 at the desired angle O depicted in FIG. 5. The angle Θ, to be described further with FIG. 6A, is defined relative to the longitudinal axis C_{L} of the main body 12 of the dental tool 10. This angle permits a dentist, hygienist, or assistant to enable viewing a patient's upper teeth without undue discomfort. However, other angles may be used, for example within a range of 10 to 60 degrees. In some embodiments of the dental tool 10 this angle may be adjustable.

FIG. 4 illustrates an exploded view of the main body portion of the embodiment shown in FIG. 2. It will be observed that the orientation of the dental tool 10 in FIG. 4 is opposite that shown in FIGS. 1, 2, and 3. In other words, the head piece 14 end of the tool (and the forward end 52 of the main body 12 in FIG. 1) is oriented to the left in the drawing in FIGS. 1, 2, and 3, and to the right in FIG. 4. This orientation of the figures is chosen to best illustrate some of the detail features of the construction of the tool. Component parts in this drawing bearing the same reference numbers as described in FIGS. 1, 2, and 3 describe the same structural components. Further, this exploded view includes fasteners such as screws 90, which are used in a number of places in assembling the parts as shown. The locations of these screw fasteners and the parts involved are clearly apparent in the drawing and will not be further described. In some applications, FDA-compliant epoxy adhesives may be used to assemble certain components together, as will be understood by persons skilled in the art.

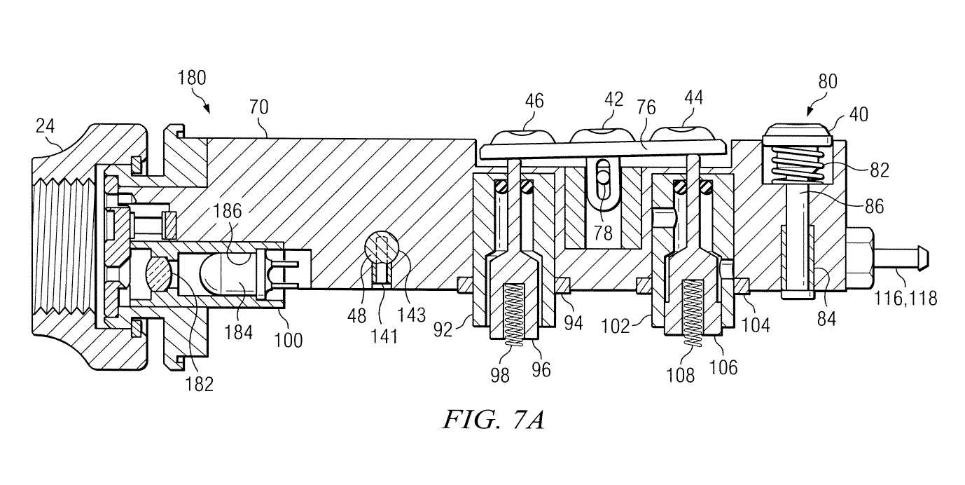

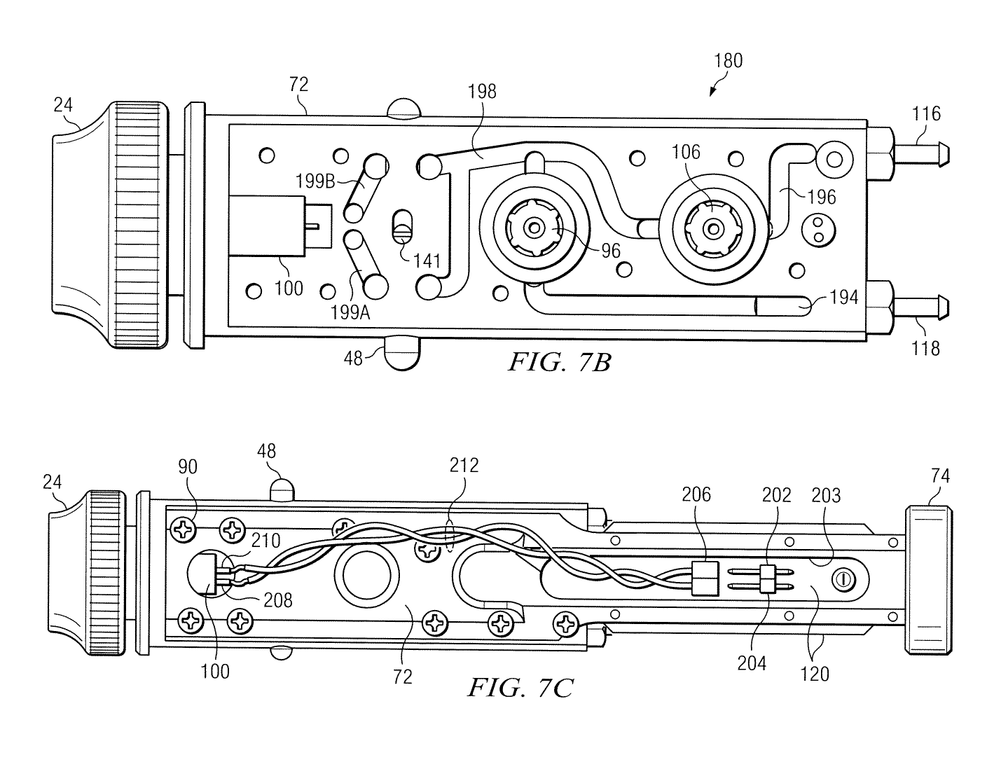

The assembly shown in FIG. 4 includes the control body 70 and its chassis 72 that are enclosed within the main body cover 20. After the main body cap 22 and the main body cover 20 are threaded onto the supply hose 18, and the fluid and air lines in the supply hose 18 are attached to the barbed fluid fittings 116, 118 in the control body 70, the main body cover 20 may be slipped over the control body 70 and secured by the main body cap 22, which may be screwed into the bracket 74. In a similar fashion, the internal passages within the control body 70 for the fluid and air 199A and 199B (See FIG. 7B to be described) may be connected through first and second junction tubes 66A and 66B inserted through passages 61 and 63 in the bulkhead 38. The junction tubes 66A and 66B may be sealed by an FDA-compliant elastomer junction tube seal 68 as shown in FIG. 4. The bulkhead 38 is retained within the forward end 52 of the control body 70 using the stainless steel retaining ring 150. The retaining collar 24 is screwed onto the (externally) threaded forward end 52 of the control body. Then, the removable head piece 14 is mated to the forward end of the main body 12 such that the fluid and light passages and tubes are aligned with the ports in the bulkhead 38 assembly, the retaining collar 24, previously installed on the forward end 52 of the control body 70 may be screwed to the threaded portion of the removable head piece.

The control body 70 contains the fluid/air poppet valve assemblies 92 through 108, the control buttons 40 through 46 and their related parts, and the light assembly 100 to be described. The control body 70 and the bracket 74 are secured to the chassis 72 using the screws 90 as shown. The chassis 72 also supports a circuit board assembly 120 that includes an electric circuit 200 (See FIG. 8) and a battery 130 (shown as battery 220 in FIG. 8) for powering the electric circuit 200 and the light assembly 100. The circuit board 120 is secured to the chassis 72 using the screws 90 as shown. The control body 70, chassis 72, and bracket 74 may be fabricated from the same stainless steel and FDA-compliant plastic materials described herein above.

The circuit board 120 includes a battery 130 mounted thereon in first and second clips 122, 132, which may be attached to the circuit board 120 with rivets 124, 134. The battery 130 may be any small battery capable of supplying at least 3.3 Volts to operate an LED as the light source for the dental tool 10. In the illustrated embodiment, which includes a regulated power supply, a Lithium-ion, rechargeable type CR123 battery rated at 3.6 Volts may be provided. The circuit board 120 may be a printed circuit board (PCB) having circuit trace contacts 228, 230 (not shown in FIG. 4, but see FIG. 8 for the circuit diagram) disposed parallel with and spaced closely to each other on the upward side of the circuit board 120 on the upper surface of the tab 136. The circuit board 120 is mounted on the upper surface 72A of the chassis 72 such that the light button actuator 86 is positioned immediately above the pair of closely spaced circuit board traces 228, 230. These contacts 228, 230 of the switch 40 shown in FIG. 8 are connected together whenever the light button actuator 86 is pressed downward into contact with the contacts 228, 230 against the tension of the return spring 82 that is seated in a recess 81 in the control body 70. The light button actuator 86 slides within a sleeve 84 disposed in the control body 70 just below the light button 40 and return spring 82. The contact closure thus provided whenever the switch button 40 is pressed connects a signal to the circuit to be described that energizes a light assembly 100.

The light assembly 100 is also depicted in FIG. 4. When assembled, it is supported within a recess in the control body 70 with a forward end butted up against the bulkhead 38. The light assembly 100 includes a small diameter tube serving as a light assembly body 186 containing a double convex lens 182 in the forward end that is butted up against the bulkhead 38 and a light emitting diode (LED) 184 disposed in the light assembly body 186 just behind the lens 182 such that the terminals of the LED 184 extend toward the PC board 120. The LED 184, preferably in this example a round, 5 mm diameter LED lamp providing a white light, is connected to the circuit board 120 through a wiring harness 212 (See FIG. 7C) routed from the terminals of a header 188 on the circuit board 120 to the first and second terminals of the LED 184. The pair of wires in the wiring harness 212, which may preferably be approximately 24 gauge stranded, insulated wire, may be dressed along and against the underside of the chassis 72 between the circuit board 120 and the light assembly 100, where the wires of the wiring harness 212 may be soldered to the terminals of the LED 184. Details of the wiring harness 212 shown in FIG. 7C to be described herein below. The circuit for driving the LED 184 is shown in FIG. 8.

The light switch assembly is controlled by the light switch button 40 and the contacts 228, 230 on the circuit board 120, which thus form a momentary switch to control the light beam of the dental tool 10. Each operation of the switch button 40 applies a ground signal to pin 2 of a PIC microcontroller U1 (See microcontroller 232 in the circuit 200 shown in FIG. 8) on the circuit board 120 to turn on a MOSFET 236 to draw current through the LED 184 in the light assembly 100 until a subsequent operation of the light switch button 40. The subsequent operation of the light switch button 40 signals the MOSFET 236 to turn OFF, opening the LED 184 circuit to extinguish the light source. In other applications, the light switch assembly may be a latching type such as, for example, a mechanical push ON, push OFF type so that the light may remain energized during use without having to hold the light switch button 40 in a depressed position. However, the PIC microcontroller 232 and MOSFET 236 combination was chosen for this application because of the mechanical simplicity and tight space requirements of the dental tool 10.

Continuing with FIG. 4, the poppet valves 92-98 and 102-108 contained within the control body 70 for controlling the flow of fluids will be described. The control body 70 may be a solid machined, cast, or molded structure of material such as alloy 304 stainless steel or nylon having bodies 92, 102 for receiving the valve poppets 96, 106 therein. The valve poppets 96, 106 are disposed within the respective valve bodies 92, 102. The valve seals 94, 104 of an FDA-compliant elastomer are also seated against valve seats 93, 103 formed respectively in the chassis 72 when the control body 70 and the chassis 72 are assembled together. The side view of the control body assembly shown in FIGS. 7A and 7B illustrate the assembled relationship of these poppet valve 92-98 and 102-108 assemblies.

Actuation of the poppet valves 92-98 and 102-108 for controlling the flow of fluids through the dental tool 10 is provided by the button assembly 76. The button assembly 76 is seated into an elongated relief 77 formed into the upper side of the control body 70. A loop 43 formed into the underside of the button assembly 76 is secured with a pin 78 that is inserted into a bore 79 passing through the control body 70 as shown. In operation, the button assembly 76 rocks about the pin 78 so that either end, 44 or 46 of the button assembly 76, when pressed, operates its respective poppet valve 92-98 or 102-108 to release a flow of fluid—typically air or water—into the removable head piece 14. If the user presses the center button 42, both poppet valves 92-98 and 102-108 may be operated to release fluids into both the first and second fluid tubes 60, 62 in the removable head piece 12. Thus, the button assembly 76 functions as a three-way control in the manner of A, B, or A and B.

The selector bar 48, for selecting the direction of fluid or air to be emitted from the fluid/light manifold 26 toward the mirror 16 or toward the patient's teeth, is installed laterally within a selector passage 141 through the side of the main body 12. The selector bar 48 is sealed in the selector passage 141 by the four elastomer O-rings 142, and secured there within by a screw 141 as shown in the exploded view of FIG. 4. In operation, the selector bar 48 is pressed from one side of the main body 12 to the opposite side to direct the flow one direction or the other. An opening 140 in a center portion of the selector bar 48 acts as a valve for directing the flow of fluid—air and/or water, for example. IN one position the opening 140 is aligned with a passage that permits the flow of fluid or air to be directed toward the mirror; in the other (second) position, the opening 140 is aligned with a passage that permits the flow of fluid or air to be directed to the patient's teeth.

FIG. 5 illustrates an internal cross section view from a first side of the embodiment of FIG. 1, showing the assembled relationship of many of the component parts illustrated in the exploded view of FIG. 4. It should be noted that the control body 70 and its chassis 72, as assembled, include the bulkhead assembly 38, the selector bar 48, the first and second poppet valve assemblies 92-98 and 102-108, the light assembly 100 and its light switch button 40 and actuator 86 assembly, and the circuit board 120 and battery 130. As enclosed in the main body cover 20, this assembly makes up the main body 12 of the dental tool 10. The main body cover 20 is secured to the control body by the main body cap 22 when it is screwed into the bracket 74 that is attached to the control body chassis 72 as described in FIG. 4. The fluid supply line 18 containing the fluid and air lines 160, 162 is connected to the barbed fluid fittings 116, 118 respectively. The barbed fluid fittings 116, 118 are screwed into the control body 70 at the location shown in FIG. 5 behind the light switch actuator 86. Finally, after the removable head piece 14 is mated to the forward end of the main body 12 such that the fluid and light passages and tubes are aligned with the ports in the bulkhead 38 assembly, the retaining collar 24, previously installed on the forward end 52 of the control body 70 may be screwed to the threaded portion of the removable head piece. The alignment of the passages is facilitated by the alignment pin 66 shown in the figure as the retaining collar 24 is drawn into a secured position. This completes the assembly of the major subassemblies of the dental tool 10.

Continuing with FIG. 5, a portion of a longitudinal axis C_{L} runs lengthwise through the body of the dental tool 10 is shown in FIG. 6A as a straight line. It will be understood that the longitudinal axis C_{L} extends through the entire dental tool 10. However, persons skilled in the art may recognize that this axis may be curved or bent at a defined angle to provide a dental tool that is balanced a certain way or formed for ease of manipulation. Such considerations may arise from the use of certain materials that are lighter than others used in the tool or, when used near one end of the tool may have a substantial affect on the center of gravity. Providing a dental tool 10 that is curved or bent at an angle along its length is among the alternative configurations contemplated in this invention. For example, in some embodiments it may be reasonable to provide a dental tool configured with the multiple functions described herein that is formed with a handle aligned away from the longitudinal axis CL instead of aligned with it. Alternatively, other embodiments, which may be intended to be supported other than by hand, may be configured to be supported by or suspended from other apparatus, thus requiring alternative forms and/or features in the tool without departing from the concepts described herein.

FIGS. 6A, 6B, and 6C depict the removable head piece 14 shown in FIG. 3 but as assembled in cross section. All of the structures identified in the FIGS. 6A, 6B, and 6C are the same ones described in FIG. 3 and bear the same reference numbers. FIG. 6A illustrates a first side cross section view of the removable head piece 14 of the embodiment of FIG. 2 showing an air and water tube 60 installed within the body of the removable head piece 14 and directed toward the mirror 16. The air and water tube 60 includes an inlet end 170 supported by the bulkhead 38 and sealed by an elastomer seal 68, and an outlet end with orifice 172 supported in the fluid/light manifold 26. Also shown in this view is an alignment pin 66 to ensure correct alignment between the removable head piece 14 and the control body 70 in the main body 12 when they are assembled together. Further, the light passage 64 to be described in FIG. 6C is also shown in profile.

Continuing with FIG. 6A, the longitudinal axis C_{L} that runs lengthwise through the body of the dental tool 10 defines a reference line for other aspects of the invention. For example, the angle Θ of the mirror 50 (and the mirror 16) relative to the longitudinal axis C_{L} of the main body 12 is shown in this illustrative example as approximately 30 degrees. This angle, in other embodiments, may be set within the range of 10 to 60 degrees. Further, in some embodiments the angle Θ may be made adjustable.

FIG. 6B illustrates a second side cross section view of the removable head piece 14 of the embodiment of FIG. 2 showing an air and water tube 62 installed within the body of the removable head piece 14 and directed toward a patient's teeth. The air and water tube 62 includes an inlet end 174 supported by the bulkhead 38 and sealed by the elastomer seal 68, and an outlet end with orifice 176 supported in the fluid/light manifold 26.

FIG. 6C illustrates a third side cross section view of the removable head piece portion of the embodiment of FIG. 2 showing a light passage 64 installed within the body of the removable head piece 14 and directed at an outlet end toward a patient's teeth. The light passage 64 includes a fiber optic bundle installed within a tube or sheath, an inlet end 178 supported by the bulkhead 38 and an outlet end with orifice 180 supported in the fluid/light manifold 26. The fiber optic bundle is preferably polished on both ends and sealed with an FDA-compliant epoxy. The alignment pin 66, provided to ensure that the passages for water, air, and light are correctly aligned when the removable head piece 14 is installed to the control body 70 is clearly shown in FIG. 6C.

FIG. 7A illustrates a side cross section view of the control body sub-assembly 180 portion of the main body 12 of FIG. 5, detached from the chassis 72 and other structures supported by the chassis 72 for clarity. FIG. 7B illustrates a bottom view of the control body sub assembly 180 shown in FIG. 7A. Structures identified in FIGS. 7A and 7B having the same reference numbers that appear in previous figures—primarily FIGS. 4 and 5—refer to the same structures. The view in FIG. 7A of the control body 70 illustrates the light assembly 100, the poppet valve assemblies 92-98 and 102-108 and the fluid control button 76, the selector bar 48 and the light switch assembly 82-86 and the light button 40.

The light assembly 100 shown in FIG. 7A includes a double convex lens 182, a light emitting diode 184, and a light assembly housing 186. The double convex lens 182 may be preferably be formed of glass or FDA-compliant plastic materials. The light emitting diode (“LED”) 184 used in the illustrative example may be a 5 mm round unit designed to emit a cool white light having a minimum color temperature of 4600° K (degrees Kelvin) and a minimum luminous intensity of approximately 20,000 mcd (millicandles) at a nominal forward current of approximately 30 mA (milliAmperes) when operated at its rated forward voltage. Persons skilled in the art will recognize that the particular choice of LED depends closely on the application and may vary depending on additional design considerations. The light assembly housing may be molded or machined of FDA-compliant plastic or alloy 304 stainless steel. The internal dimensions of the housing 186 may be calculated to allow a nominal press fit of the lens 182 and LED 184.

The poppet valve assemblies shown in FIGS. 7A and 7B control the flow of fluid and or air according to how the button 76 is pressed, as described previously in FIG. 4. When released, the button 76 blocks the flow of fluid (e.g., water) and or air; when one end (either 44 or 46 of the button 76 is pressed the fluid or air in the associated passage 194 or 196 machined in the control body 70 is allowed to flow through the respective poppet valve to a common passage 198, and thence through bypass passages 199A or 199B and the bulkhead 38 to a corresponding tube 60, 62 in the removable head piece 14. The passages 194, 196, 198, 199A and 199B may be preferably machined in the control body (such as by routing and drilling, for example) similar to the valve body used in a typical automatic transmission for automobiles. When the button 76 is pressed at its midpoint 42 (or, alternatively, at both ends 44, 46 simultaneously) fluid and or air will both be admitted to the common passage 198 for dispersal through the passages 198 and 199 and fluid tubes 60, 62. The button 76, retained in the control body 70 by a pivot pin 78 positioned in a slot 43 in the button 76, is urged into its released position by first 98 and second 108 poppet springs as shown in FIG. 4.

Continuing with FIG. 7A and 7B, the selector bar 48, which slides laterally in a bore 79 passing through the control body 70, provides control to cause the fluid or air selected by one of the poppet valve assemblies 92-98 or 102-108 to be directed to one or the other of the two fluid tubes 60, 62, depending whether the user wishes to direct the fluid or air toward the mirror 16 or to the patient's teeth. The selector bar 48 is retained within its bore 79 by a retaining screw 141. The light switch assembly 80 includes the light button 40, return spring 82, sleeve 84, and switch actuator 86 contained within a bore 81 in the control body 70. Also depicted in FIGS. 7A and 7B are the first and second barbed fluid fittings 116, 118 that are screwed into threaded bores in the control body 70. The barbed fluid fittings 116 and 118 provide connection points for the fluid and air lines 160 and 162 within the supply line 18 shown in FIG. 5.

FIG. 7C illustrates an underside view of the control body chassis 72 assembled with a retaining collar 24 and wiring harness 212 for the light assembly 100. The chassis 72 is secured to the control body 70 with screws 90, several of which are not illustrated for clarity of the wiring harness 212 that is dressed along the surface of the chassis 72 as shown. The chassis 72 is depicted between the retaining collar 24 and the bracket 74. The circuit board 120 is also visible in this view through the elongated opening 203 in the chassis 72. The wiring harness 212 connects between first and second terminal pins 202, 204 that are positioned in the opening 203 and connect to the circuit board 120. The connection between the individual wires of the harness 212 and the terminal pins 202, 204 is via a receptacle 206. The opposite ends of the wires in the harness 212 may be soldered to the first 208 and second 210 pins of the LED 184 or connected using a receptacle (not shown) suitable for the purpose as readily understood by persons skilled in the art. The LED 184 is disposed within the light assembly 100 and thus not visible in this view, except for the first and second pins 208, 210. Please refer to FIGS. 5 and 7A for the detail of the light assembly. The first and second wires of the harness 212 may preferably be twisted to assist in maintaining a neat dress against the surface of the chassis 72.

FIG. 8 illustrates an electrical circuit diagram 200 of the light source portion of the embodiment of FIGS. 4 and 5. A voltage regulator 224 powered by a battery 220 having an output 222 applied to an input terminal of the voltage regulator is provided to produce a stable voltage at an output terminal 226. The battery voltage may be rated at 3 to 9 Volts, depending on the input voltage requirements of the voltage regulator 224 and the type available that meets the small physical size and current requirements. In the present embodiment a 3.6 Volt Lithium ion rechargeable battery may be used. The voltage at the output terminal 226 “3.3 Vcc” may preferably be 3.3 Volts in the illustrated example, which is sufficient to power the LED 184 light source and the PIC microcontroller 232 programmed to control the current supplied to the LED 184 light source and to provide a debounce function to ensure reliable operation of the light switch 40. Light switch 40 includes first and second terminals, which may be first 228 and second 230 PC board traces disposed in an extension tab portion 136 of the circuit board 120 as shown in FIG. 4. The PC board traces 228 and 230 are not shown in FIG. 4 but are represented by the terminal symbols 228, 230 in FIG. 8. The light switch 40 is connected to a GP5 terminal of the PIC microcontroller 232, and to a pull up resistor 248 such that the GP5 pin of the microcontroller 232 is pulled HIGH (toward the 3.3 Volt supply) except when the switch 40 is activated by pressing on the light switch button 40 to pull the GP5 terminal LOW to energize the light source. This action causes the gate drive signal to be output from terminal GPO of the microcontroller 232 on signal line 238 to the gate of the MOSFET switch 236, causing it to conduct current through the LED 184, which is connected to the regulated supply voltage through a resistor 240. In the illustrated embodiment, one suitable LED is a type C503D-WAN available from Cree, Inc, Durham, N.C. 27703.

Continuing with FIG. 8, the 3.3 Volt regulated supply is shown connected to a V_{DD} terminal pin of the microcontroller 232. Further, a second pullup resistor 246 is shown connected to an MCLR* terminal of the microcontroller 232. A voltage divider formed by two equal valued resistors 242, 244 provides a voltage of one half the regulated supply voltage to a GP4/AN3 terminal of the microcontroller. The microcontroller 232 is programmed to cause the LED 184 to blink, indicating low battery voltage, if this value equal to one-half the supply voltage falls below an internal reference level. In the illustrated embodiment one suitable microcontroller may be a type PIC12C671.

Persons skilled in the art will understand that numerous variations are possible or that additional features may be included in the illustrated circuit to adapt it to particular applications. The present circuit is illustrated to provide one generalized method of replicating the present invention. In the illustrated embodiment a separate ON-OFF switch to control application of power to the circuit from the battery is not included because the circuit draws negligible current when the dental tool 10 is not in use, and the circuit is thus always in a stand-by, ready to use condition as long as the battery provides sufficient voltage. A quick tap on the button 40 to test the light can be used to confirm the readiness of the dental tool 10 for use. This particular approach eliminates a separate on-off switch, which is both a cost and an ergonomic issue in the use of the tool.

While the invention has been shown in only one of its forms, it is not thus limited but is susceptible to various changes and modifications without departing from the spirit thereof.

What is claimed is:

1. For use with a dental mirror tool having a body and a mirror attached to an end thereof and disposed at an angle to a longitudinal axis of the tool body, an apparatus, comprising:
a manifold disposed on the tool body proximate the mirror;
a plurality of output ports disposed in an outlet side of the manifold, the ports positioned to emit streams of light and fluid and air toward a predetermined target; and
a selection system disposed in the tool for selecting the light, fluid, or air emitted toward the predetermined target.

2. The apparatus of claim 1, wherein the plurality of output ports disposed in an outlet side of the manifold are positioned to emit streams of light or fluid or air toward a target region of a patient's mouth.

3. The apparatus of claim 2, wherein the plurality of output ports includes a suction port positioned to direct a suction stream toward a target region of a patient's mouth.

4. The apparatus of claim 1, wherein the plurality of output ports disposed in an outlet side of the manifold are positioned to emit streams of light or fluid or air toward a point on the surface of the mirror.

5. The apparatus of claim 1, further comprising:
a plurality of internal passages disposed within the tool body for conducting the light and fluid and air to respective ones of the plurality of output ports.

6. The apparatus of claim 5, the selection system comprising:
one or more controls for controlling the conduction of the respective light and fluid and air in the passages and emission thereof from the respective output ports.

7. The apparatus of claim 5, the selection system comprising:
in operative association with an external button disposed on the tool body, a poppet valve disposed within the respective passages for controlling the conduction of fluid and air.

8. The apparatus of claim 1, the selection system comprising:
an electric circuit associated with a light source within the tool body for controlling the production of light.

9. The apparatus of claim 8, further comprising:
a switch operatively associated with an external button disposed on the tool body for controlling the emission of light.

10. A dental tool, comprising:
a body configured as an elongated handle having an angled mirror attached thereto;
a manifold disposed along the body; and
a plurality of fluid, air, and light emission ports disposed in a forward side of said manifold and operable to be aimed toward the mirror or a region of a patient's mouth.

11. The apparatus of claim 10, further comprising:
a plurality of internal passages disposed within the body for conducting the light and fluid and air to respective ones of the plurality of output ports.

12. The apparatus of claim 10, further comprising:
an internal passage disposed within the body for conducting an air suction stream via an air passage and emission port directed toward a region of the patient's mouth.

13. The dental tool of claim 10, further comprising:
a selection system disposed in the tool for selecting fluid, air, or light emitted toward the mirror.

14. The apparatus of claim 13, the selection system comprising:
one or more controls for controlling the conduction of the respective light and fluid and air in the passages and emission thereof from the respective output ports.

15. The apparatus of claim 13, the selection system comprising:
in operative association with an external button disposed on the tool body, a poppet valve disposed within the respective passages for controlling the conduction of fluid and air.

16. In a single dental tool, a method for directing streams of light, air, and fluid toward a target during a dental procedure, comprising the steps of:
attaching a manifold having output orifices for air, fluids, and light to a body of said tool;
connecting said orifices to sources of said air, fluid, and light; and
controlling a substance emitted from said orifices to be directed toward said target.

17. The method of claim 16, further comprising the steps of:
conducting said light and fluids via respective passages; and
providing respective control buttons for independently controlling the conduction of said light and fluid through said passages.

18. The method of claim 16, wherein said target is a dental mirror attached to said dental tool.

19. The method of claim 16, wherein said target is a patient's tooth, and said method includes the step of:
providing suction via a said orifice connected to a source of vacuum.

20. A dental apparatus, comprising:
a cylindrical body having an arm extending from a first end of the body along an axis parallel to a longitudinal axis of the body;
a mirror surface attached to a distal end of said arm and disposed at a predetermined angle with respect to said longitudinal axis;
a manifold disposed on a side of said arm intermediate said first end of said body and said mirror surface; wherein
said manifold includes a plurality of outputs for emitting streams of light, air, and fluid toward predetermined locations in a patient's mouth.

21. The dental apparatus of claim 20, further comprising:
an output for providing suction directed toward predetermined locations in a patient's mouth.

22. A manifold for attaching to a handle of a dental tool having an angled mirror disposed on a first end thereof, the manifold comprising:
a plurality of air, fluid, and light emission ports aimed at predetermined regions relative to the dental tool.
